(19) United States
(12) Patent Application Publication
(10) Pub. No.: US 2024/0122858 A1
(43) Pub. Date: Apr. 18, 2024

(54) MODIFIED-RELEASE DOSAGE FORMS OF RUXOLITINIB

(71) Applicant: SLAYBACK PHARMA LLC, Princeton, NJ (US)

(72) Inventors: Paras P. JAIN, Amaravati (IN); Krishna Mohan LAKSHMIPATHULA, Hyderabad (IN); Somnath Devidas NAVGIRE, Hyderabad (IN); Hanimi Reddy BAPATU, Hyderabad (IN); Sandeep JAIN, Sagar (IN); Sumitra Ashokkumar PILLAI, Hyderabad (IN); Praveen Kumar SUBBAPPA, Princeton, NJ (US)

(73) Assignee: SLAYBACK PHARMA LLC, Princeton (NJ)

(21) Appl. No.: 18/532,596

(22) Filed: Dec. 7, 2023

Related U.S. Application Data
(63) Continuation of application No. 18/075,150, filed on Dec. 5, 2022, now abandoned.

(30) Foreign Application Priority Data
Dec. 7, 2021 (IN) 202141056879

(51) Int. Cl. A61K 9/20; A61K 9/28; A61K 31/519

(52) U.S. Cl. CPC A61K 9/2077; A61K 9/2013; A61K 9/2018; A61K 9/2031; A61K 9/2054; A61K 9/28; A61K 31/519

(57) ABSTRACT

Modified-release pharmaceutical compositions of ruxolitinib or a pharmaceutically acceptable salt thereof are disclosed. Preferably, the invention relates to oral modified-release pharmaceutical compositions of ruxolitinib, which enable once-daily administration. Oral modified-release compositions of ruxolitinib, methods for their administration, processes for their preparation, and use of these compositions for treatment of diseases treatable by ruxolitinib are also described.

### CROSS-REFERENCE TO RELATED APPLICATIONS

This application is a Continuation of co-pending application Ser. No. 18/075,150, filed on Dec. 5, 2022, which claims priority to Indian Application No. IN 202141056879, filed on Dec. 7, 2021, the contents of which are incorporated herein by reference in their entirety.

### FIELD OF THE INVENTION

The present invention relates to modified-release pharmaceutical compositions of ruxolitinib or its pharmaceutically acceptable salts thereof. Preferably, the invention relates to oral modified-release pharmaceutical compositions of ruxolitinib, for once-daily administration.

The present invention further relates to oral extended-release compositions of ruxolitinib, methods for their administration, processes for their preparation, and use of these compositions for treatment of diseases treatable by ruxolitinib.

### BACKGROUND OF THE INVENTION

Ruxolitinib is a potent Janus kinase (JAK) inhibitor, selectively inhibits JAK1 and jAK2 receptors, which mediate the signaling of a number cytokines and growth factors that are important for hematopoiesis and immune function. Dysregulated JAK1 and JAK2 signaling causes unchecked activation of various cytokines and growth factors, which leads to myeloproliferative disorders (MPDs), including polycythemia vera, essential thrombocythemia and primary myelofibrosis. By inhibiting JAK1 and JAK2, ruxolitinib blocks the dysregulated cell signaling pathways and prevents abnormal blood cell proliferation. Chemically, this compound belongs to the class of organic compounds known as pyrrolo-[2,3-d]pyrimidines. The chemical name of ruxolitinib is (3R)-3-cyclopentyl-3-[4-(7H-pyrrolo[2,3-d]-pyrimidin-4-yl)-pyrazol-1-yl]-propanenitrile phosphate and its chemical structure is represented as follows:

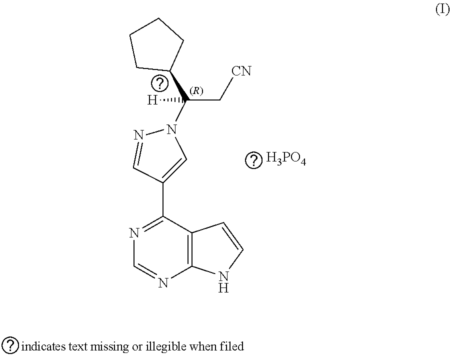

Ruxolitinib is currently approved and marketed in United States under the brand name JAKAFI® for the treatment of myelofibrosis, polycythemia vera, acute graft-versus-host disease (aGVHD) and chronic graft-versus-host disease (cGVHD). JAKAFI® (New Drug Application (NDA) Number 202192 and National Drug Code (NDC) Numbers 50881-005; 50881-010; 50881-015; 50881-020 and 50881-025) is commercially available in the form of immediate-release tablets in five different strengths. Each tablet contains 6.6 mg, 13.2 mg, 19.8 mg, 26.4 mg, or 33 mg of ruxolitinib phosphate equivalent to 5 mg, 10 mg, 15 mg, 20 mg, or 25 mg of ruxolitinib free base, respectively, together with microcrystalline cellulose, lactose monohydrate, magnesium stearate, colloidal silicon dioxide, sodium starch glycolate, povidone and hydroxypropyl cellulose as inactive ingredients. The recommended starting dose of ruxolitinib is based on the platelet count of a patient. A complete blood count (CBC) and platelet count is performed before initiating therapy, every 2 to 4 weeks until doses are stabilized. In case of myelofibrosis, the recommended starting dose can be as high as 20 mg orally twice daily, only if platelet count reads greater than 200×10⁹/L. For polycythemia vera, the recommended starting dose of ruxolitinib is 10 mg twice daily. For aGVHD and cGVHD, the recommended starting dose of ruxolitinib is 5 mg and 10 mg orally twice daily respectively.

Ruxolitinib, as a free base, is sparingly soluble in aqueous buffers and has a pH-dependent solubility. However, Ruxolitinib phosphate has been designated a Class 1 compound in the Biopharmaceutical Classification System (BCS) based on its high aqueous solubility (over a pH range of 1-8), high permeability and rapid dissolution characteristics. Following oral administration, JAKAFI® (Ruxolitinib immediate release tablets) undergoes rapid absorption and peak plasma concentrations are reached within one hour after administration, with elimination half-life of approximately 3 hours.

Treatment with ruxolitinib can cause hematological adverse drug reactions, including thrombocytopenia, anemia and neutropenia. A complete blood count, including a white blood cell count differential, must be performed before initiating therapy with ruxolitinib. Treatment should be discontinued in patients with platelet count less than 50,000/mm³ or absolute neutrophil count less than 500/mm³. It has been observed that patients with low platelet counts (<200,000/mm³) at the start of therapy are more likely to develop thrombocytopenia during treatment. Thrombocytopenia is generally reversible and is usually managed by reducing the dose or temporarily withholding ruxolitinib treatment. Dose modifications or interruption for patients developing anemia may also be considered. Patients with a hemoglobin level below 10.0 g/dL at the beginning of the treatment have a higher risk of developing a hemoglobin level below 8.0 g/dL during treatment compared to patients with a higher baseline hemoglobin level. More frequent monitoring of hematology parameters and of clinical signs and symptoms of ruxolitinib-related adverse drug reactions is recommended for patients with baseline hemoglobin below 10.0 g/dL. Similarly, neutropenia (absolute neutrophil count <500) was generally reversible and was managed by temporarily withholding ruxolitinib.

According to the above recommendations, patients undergoing treatment with ruxolitinib require careful analysis of their haematological parameters, before and during the course of dosage regimen. The health care professionals and caregivers thus, have to constantly monitor the patients, report the adverse events and adjust the ruxolitinib dosage accordingly, in order to cater to the needs of patients.

As stated earlier, the absorption of JAKAFI® (Ruxolitinib immediate release tablets) is rapid, with peak plasma concentrations occurring within 1-2 hours following oral administration, with elimination half-life of approximately 3 hours. These properties lead to administration of twice daily doses of JAKAFI® (Ruxolitinib immediate release tablets) for optimal treatment. However, administration of ruxolitinib medicament in twice daily doses does not provide patient compliance. Further, each administration of JAKAFI® (Ruxolitinib immediate release tablets) is associated with a peak plasma concentration of the drug, and the fluctuations associated with the peaks and valleys of blood plasma levels of the drug is not desirable, as it contributes to unwanted serious side effects like thrombocytopenia (low platelet count) and anaemia (low haemoglobin). The incidence of side effects of ruxolitinib directly correlates with the maximum plasma concentration i.e. Cₘₐₓ, while the efficacy of ruxolitinib is predominantly associated with the total amount of ruxolitinib absorbed after repeated administration i.e. area under the curve (AUC).

Thrombocytopenia is dose-dependent and considered the dose-limiting toxic effect. Thrombocytopenia and anaemia side effects lead to dose-reductions, treatment interruption or transfusion of platelets and or blood. The other serious condition is symptom exacerbation following treatment interruption or discontinuation. Following discontinuation of ruxolitinib, symptoms from myeloproliferative neoplasms may return to pre-treatment levels over a period of approximately one week.

Various attempts have been made in the past to formulate ruxolitinib in the form of modified-release dosage forms.

U.S. Patent Publication No. 20150246043 disclose modified-release dosage forms suitable for oral administration, comprising ruxolitinib, non-erodible material and a pore-forming material, providing a drug release of less than 10% within 2 hours and more than 80% within 3 to 12 hours, preferably between 4 and 8 hours.

U.S. Pat. Nos. 10,874,616 & 10,166,191 disclose oral sustained-release dosage forms comprising ruxolitinib and a sustained release matrix former such as hydroxypropyl methylcellulose (HPMC, hypromellose).

There exists a need for developing novel modified-release dosage forms of ruxolitinib suitable for once-daily oral administration, preferably, an oral dosage form having acceptable content-uniformity, solubility, dissolution profile, predictable and reproducible dissolution rates, stability and bioavailability. Such an oral dosage form should be producible in a large scale in an economic beneficial way and should be able to provide desirable plasma levels of ruxolitinib.

There also exists a need in the art to provide a monolithic, matrix-based extended-release oral dosage form which comprises polyethylene oxide as the sole release-controlling agent, providing for sustained release of ruxolitinib, enabling once-a-day administration.

There also is a need in the art to provide a monolithic, matrix-based extended-release oral dosage form that does not contain hydroxypropyl methylcellulose (HPMC) as release-controlling agent, providing for sustained release of ruxolitinib, enabling once-a-day administration.

The present invention fulfils this need by developing oral extended-release dosage forms of ruxolitinib suitable for once-daily administration to achieve an improved standard of patient care.

### SUMMARY OF THE INVENTION

Certain embodiments of the present invention relate to a monolithic, matrix-based extended-release pharmaceutical composition suitable for once-daily administration, said composition including: a) ruxolitinib phosphate; and b) a release-controlling polymer which is polyethylene oxide; wherein not less than 20% of total amount of ruxolitinib is released from said composition within 2 hours, and not more than 60% of total amount of ruxolitinib is released from said composition within 3 hours as determined by USP dissolution apparatus II (paddle) at 50 rpm in 500 mL of 0.1N HCl as dissolution medium.

Certain embodiments of the present invention relate to a monolithic, matrix-based extended-release pharmaceutical composition suitable for once-daily administration, said composition including: a) ruxolitinib phosphate; and b) a release-controlling polymer which is polyethylene oxide; wherein not less than 20% of total amount of ruxolitinib is released from said composition within 2 hours as determined by USP dissolution apparatus II (paddle) at 75 rotations per minute (rpm) in 500 mL of 0.1N HCl as dissolution medium and wherein not more than 70% of total amount of ruxolitinib is released from said composition within 3 hours as determined by USP dissolution apparatus II (paddle) at 75 rotations per minute (rpm) in 900 mL of re-buffered dissolution medium to pH 6.8.

In certain embodiments of the composition as described above, the composition further includes at least one stabilizing agent.

In certain embodiments of the composition as described above, the level of total impurities in said composition is less than 1.5% w/w when stored at 40° C./75% RH or 25° C./60% RH for at least 3 months.

In certain embodiments of the composition as described above, the composition does not include hydroxypropyl methyl cellulose as release-controlling polymer.

In certain embodiments of the composition as described above, the polyethylene oxide is a sole release-controlling polymer.

In certain embodiments of the composition as described above, the composition includes an intra-granular phase containing at least said ruxolitinib phosphate and said polyethylene oxide; and an extra-granular phase containing at least one additional excipient selected from diluents, binders, chelating agents, coating agents, disintegrating agents, lubricants, glidants, colorants, surfactants, plasticizers and mixtures thereof.

In certain embodiments of the composition as described above, said intra-granular phase further includes microcrystalline cellulose.

In certain embodiments of the composition as described above, said extra-granular phase includes magnesium stearate as a lubricant.

In certain embodiments of the composition as described above, the composition further includes a diluent selected from the group consisting of microcrystalline cellulose, silicified microcrystalline cellulose, calcium carbonate, calcium sulfate, cellulose powdered, dextrates, dextrins, dextrose, fructose, kaolin, lactitol, lactose, starch, starch pre-gelatinized and sucrose.

In certain embodiments of the composition as described above, the amount of said diluent ranges from about 60% to about 95% w/w of total composition.

In certain embodiments as described above, said composition is a tablet.

In certain embodiments of the composition as described above, said composition is prepared using wet granulation.

In certain embodiments of the composition as described above, the ruxolitinib phosphate is present in an amount of 13.2 mg, or 26.4 mg, or 39.6 mg, or 52.8 mg, or 66 mg, or 79.2 mg.

Certain embodiments of the present invention relate to a method of treating a condition selected from myelofibrosis, polycythemia vera, aGVHD, and cGVHD, said method including administering the composition as described above to a patient in need thereof.

### DETAILED DESCRIPTION OF THE INVENTION

Unless defined otherwise, all the technical and scientific terms used herein have the same meanings as commonly known by a person skilled in the art. In case of conflict, the definitions provided herein will prevail. Unless specified otherwise, all the percentages, portions and ratios in the present invention are on weight basis.

The term “pharmaceutically acceptable” substances mean those, which, according to a common medical judgment, are those that are commonly known as suitable for use in pharmaceutical products.

By “effective amount” or “therapeutically effective amount” is meant the amount of a drug sufficient to treat, prevent, or ameliorate a condition in a subject or patient. The effective amount of ruxolitinib or pharmaceutically acceptable salt thereof, used to practice the present invention for therapeutic management of a condition may be determined and adjusted by a person of ordinary skill to provide the appropriate amount and dosage regimen, e.g., depending upon one or more of the manners of administration, the age, body weight, sex, and/or general health of the patient.

The term “subject” refers to an animal, including a human or non-human. The terms patient and subject may be used interchangeably herein.

The terms “stable” and “stability” mean that the evolution of the product with time and/or under specific environmental conditions (i.e., temperature, humidity, etc.) has no significant effects on its quality, safety and/or efficacy for a given time period. It can be measured through the formation of degradation products (impurities), variation of pH, appearance (precipitation), microbial growth, and/or color. The term “stable” indicates both chemical and physical stability. The term “stable” can further mean as no more than about 5% loss of ruxolitinib under typical commercial storage conditions. Preferably, formulations of the present inventions will have no more than about 3% loss of ruxolitinib, more preferably, no more than about 2% loss of ruxolitinib, more preferably, no more than about 1.5% loss of ruxolitinib under typical storage conditions (i.e., 25° C./60% RH or 40° C./75% RH or 40° C./25% RH). Preferably, formulations of the present inventions will have level of total impurities in said composition is less than 1.5% w/w when stored at 40° C./75% RH or 25° C./60% RH for at least 3 months.

The term “any person” refers to any human being capable of administering a dose of ruxolitinib composition, including physicians, healthcare professions, nurse, pharmacist, pharmacy technicians and patient.

In the present invention, the terms “release-controlling agent”, “controlled release agent”, and “agent capable of retarding release” are used interchangeably and describe an excipient present in a pharmaceutical formulation, wherein said agent is capable of retarding the release of the active ingredient from the formulation. For the purpose of this invention, the terms relate to any material, used for controlling the release of an active ingredient from the formulation.

Modified-release pharmaceutical compositions are sometimes classified as “matrix”, “reservoir”, or “osmotic” mechanisms (or systems), depending on type of rate controlling mechanism employed in the composition.

Matrix-release mechanisms: Active ingredient is incorporated into a release-controlling agent to form polymeric matrix. Release-controlling agent can be aqueous-erodible or water-swellable or aqueous-soluble or may require presence of an acid or base to ionize the polymeric matrix sufficiently to cause erosion or dissolution. When contacted with the aqueous environment, the erodible polymeric matrix imbibes water and forms an aqueous-swollen gel or “matrix” that entraps active ingredient. The aqueous-swollen matrix gradually erodes, swells, disintegrates, disperses or dissolves in the environment of use, thereby controlling release of active ingredient to the environment of use.

The term “immediate release,” as used herein, refers to release of at least 85% of a drug in about one hour.

The terms “extended-release”, “extended-release form”, or “extended-release dosage form” are widely recognized in the art of pharmaceutical sciences as systems that maintains therapeutic blood or plasma or tissue levels of a drug for an extended period. An extended-release dosage form potentially provides greater effectiveness in the treatment of chronic diseases or conditions; greater convenience; reduces side effects and provides higher levels of patient compliance or therapeutic performance due to a simplified dosage schedule, compared with those of immediate-release drugs. Extended-release pharmaceutical products are formulated to release the active ingredient gradually and predictably over an extended time period, such as a 12-hour period or 24-hour period.

The present application relates to extended-release compositions of ruxolitinib, suitable for oral administration, wherein said compositions release ruxolitinib by non-osmotic mechanism.

The term “Cₘₐₓ” as used herein, refers to a maximum concentration of a drug in blood, serum, a specified compartment or test area of a subject between administration of a first dose and administration of a second dose. The term Cₘₐₓ could also refer to dose normalized ratios if specified. As used herein, “C₁₂ₕ” refers to the plasma concentration measured at 12 hours from administration.

The term “AUC_{0-∞},” means the area under a plasma drug concentration-time curve from time point of 0 to infinity after drug administration, and the term “AUC₀₋ₜ” means the area under a plasma drug concentration-time curve from time point of 0 to t after drug administration.

“Bioequivalence” refers to the absence of a significant difference between the bioavailability, i.e., the mean ratio of AUC (over 24 hours) and the mean ratio of Cₘₐₓ is within 80% to 125% between two pharmaceutical drug products (e.g., a test composition and a reference composition) over the course of a period of time, at the same dose and under the same conditions. The determination of whether or not a test composition is bioequivalent to a reference composition is determined by performing a study, referred to as a bioequivalence or comparative bioavailability study, in a group of subjects under controlled conditions.

The term “bioavailability” denotes the degree to which a drug substance becomes available to the target tissue after administration.

Unless specified otherwise, all the percentages, portions and ratios in the present invention are on weight basis.

The present application relates to extended-release compositions of ruxolitinib, suitable for oral administration, wherein ruxolitinib is present in an amount of 1 mg or more.

In particular, the present invention provides extended-release compositions of ruxolitinib for oral administration having dosing range from about 1 mg to about 300 mg, about 2 mg to about 200 mg, about 3 mg to about 150 mg, about 4 mg to about 100 mg, most preferably about 5 mg to about 60 mg of ruxolitinib on free basis. In preferred embodiments, dose of ruxolitinib ranges from about 10 to 60 mg on a free base basis. The dosage form can contain ruxolitinib, or a pharmaceutically acceptable salt thereof, in an amount of about 10 to about 60 mg, about 10 to about 40 mg, about 20 to about 40 mg, or about 20 to about 30 mg on a free base basis. In some embodiments, the dosage form contains about 10 mg, about 12.5 nag, about 20 mg, about 25 mg, about 30 mg, about 37.5 mg, about 40 mg, about 50 mg, or about 60 mg on a free base basis. The phrase “on a free base basis” indicates that the amount of ruxolitinib or salt thereof in the dosage form is measured based on the molecular weight of ruxolitinib free base only, even when the actual active ingredient is a salt of ruxolitinib having a different molecular weight than the free base. For example, the conversion factor for ruxolitinib phosphate salt to free base is 0.7575. Preferably, the doses are 10, 20, 30, 40 and 50 mg, on free base basis or 13.2 mg, 26.4 mg, 39.6 mg, 52.8 mg and 66.0 mg of ruxolitinib phosphate respectively.

Another embodiment provides a method for treating steroid-refractory acute graft-versus-host disease (aGVHD), chronic graft-versus-host disease (cGVHD), pancreatic cancer, acute lymphoblastic leukemia (ALL) and myeloproliferative disorders (MPDs) such as polycythemia vera (PV), essential thrombocythemia (ET), myelofibrosis with myeloid metaplasia (MMM), primary myelofibrosis (PMF), chronic myelogenous leukemia (CML), chronic myelomonocytic leukemia (CMML), intermediate or high-risk myelofibrosis, primary myelofibrosis (PMF), post polycythemia vera/essential thrombocythemia myelofibrosis (Post-PV/ET MF), post-essential thrombocythemia myelofibrosis (Post-ET) and polycythemia vera myelofibrosis (Post-PV MF), hypereosinophilic syndrome (HES), and systemic mast cell disease (SMCD) by orally administering therapeutically effective amount of ruxolitinib extended-release composition.

In an embodiment, extended-release composition of the present invention comprises a) ruxolitinib phosphate; and b) at least one release-controlling agent. In a further embodiment, the release-controlling agent may be a polymer or non-polymer. In a preferred embodiment, the release-controlling agent may be a polymer. In a further embodiment, the polymeric release-controlling agent may be pH-dependent or pH-independent in nature or combinations thereof.

In an embodiment, the amount of release-controlling agent used in the formulation may vary depending upon the pharmaceutically active agent employed and the degree of extended-release desired. The release-controlling agent contained in the tablet ranges from about 5 to about 80 wt. %, preferably from about 10 to about 50 wt. %, most preferably from about 15 to about 40 wt. %, based upon the total weight of the formulation. If too little release-controlling agent is used, the formulations may break up during the passage down the gastrointestinal tract and this, in turn, may lead to a premature release of a large portion of the content of the drug which is called dose-dumping. If too much release-controlling agent is used, there is a risk that some of the drug will be encapsulated and not released from the tablet. Weight percentage of release-controlling agent plays a critical role to resist dose-dumping and to avoid complete drug encapsulation.

In one embodiment, the non-polymeric release-controlling agents that may be employed in the composition of the present invention include suitable waxes such as carnauba wax, candelilla wax, spermaceti, bees wax, montan wax, microcrystalline wax, lecithin, and paraffin wax or combinations thereof.

In one embodiment, the release-controlling agent is not hydroxypropyl methylcellulose (HPMC).

In another embodiment, the polymeric release-controlling agents that may be employed in the compositions of the present invention include poly(alkylene oxide) derivatives.

Poly(alkylene oxide) derivatives include, but are not limited to, polymers which contain as a unit, ethylene oxide, propylene oxide, ethylene oxide, or propylene oxide. These polymers may consist entirely of any of the above units (as a monomer), combinations of any of the above units, such as a copolymer. In one embodiment, it is a block copolymer in which one of the repeating units consists of ethylene oxide, propylene oxide, ethylene oxide, or propylene oxide. In one embodiment, weight average molecular weight of polyethylene oxide ranging from about 200,000 to about 11,000,000.

In a preferred embodiment, the release-controlling agent employed in the present invention is poly(alkylene oxide) derivative such as poly(ethylene oxide), which is a matrix former. The term “polyethylene oxide,” or “PEO” refers to a polyethylene oxide polymer that has a wide range of molecular weights. PEO is a linear polymer of unsubstituted ethylene oxide and has a wide range of viscosity-average molecular weights. Commercially available swellable polymers include POLYOX™303 (Poly(ethylene oxide), molecular weight 7,000,000); POLYOX™ WSR N-12K (Poly(ethylene oxide), molecular weight 1,000,000), POLYOX™ WSR N-60K (Poly(ethylene oxide), molecular weight 2,000,000), POLYOX™ WSR 301 (Poly(ethylene oxide, molecular weight 4,000,000), molecular weight 4,000,000), POLYOX™ WSR Coagulant, POLYOX™ WSR 303, POLYOX™ WSR 308, NFgrade™ (Poly(ethylene oxide) molecular weight 1,000,000); POLYOX™ WSR 1105 LEO (Poly(ethylene oxide), molecular weight 900,000), POLYOX™ WSR N80™ (Poly(ethylene oxide), molecular weight 200,000); POLYOX™ WSR N750 (Poly(ethylene oxide), molecular weight 300,000). In a more preferred embodiment, PEO material suitable to use in the present invention include, but are not limited to, PEO with an average molecular weight selected from at least, 200,000, at least 5,000,000, at least 1,000,000, at least 2,000,000, at least 4,000,000, at least 6,000,000, at least 7,000,000, at least 8,000,000, at least 9,000,000 or at least 10,000,000.

The pharmaceutical composition of the present invention further also includes other pharmaceutically acceptable excipients such as diluents, binders, disintegrants, lubricants, glidants, granulating agents, anti-adherent agents, coloring agents, chelating agents, stabilizing agents, coating agents and mixtures thereof. A skilled artisan in the art would know what other pharmaceutically acceptable excipients could be included in the formulations according to the invention. The choice of excipients would depend on the characteristics of the compositions and on the nature of other pharmacologically active compounds in the formulation.

The term “diluent” as used herein is defined as an agent used as filler in order to achieve the desired tablet volume or weight. The diluent may be present in the pharmaceutical composition in the form of a single compound or in the form of a mixture of compounds. If the properties of the diluent are enough, it could also play a functional role in the formulation and one could get rid of one or several other specific processing aid excipients. Suitable diluents include, but are not limited to, microcrystalline cellulose, silicified microcrystalline cellulose, calcium carbonate, calcium sulfate, cellulose powdered, dextrates, dextrins, dextrose excipients, fructose, kaolin, lactitol, lactose, starch, starch pregelatinized, sucrose, sugar compressible, sugar confectioners and the like. The diluent concentration ranges from about 5% to about 95% w/w of total composition, preferably from about 60% to about 95%.

The term “binder” as used herein is defined as an agent used to increase the cohesion of the granules during the compression, in order to obtain tablets with a defined hardness, or to act as processing aid during a granulation process. The binder may be present in the pharmaceutical composition in the form of a single compound or in the form of a mixture of compounds. The binder may be present in the pharmaceutical composition in the intra-granular portion or dispersed/dissolved in the granulating fluid. Suitable binders include, but are not limited to, methyl cellulose, hydroxypropyl cellulose, polyvinyl pyrrolidone, microcrystalline cellulose, gelatin, gum arabic, ethyl cellulose, polyvinyl alcohol, pullulan, pregelatinized starch, agar, tragacanth, sodium alginate, propylene glycol and the like. The concentration of binder ranges from about 1% to about 50% w/w of total composition, preferably from about 10% to about 30% w/w of total composition.

The term “disintegrant” as used herein refers to any material that has wicking and/or swelling properties when it comes in contact with water. Suitable disintegrating agents include croscarmellose sodium, low substituted hydroxypropyl cellulose (L-HPC), sodium starch glycollate, carboxymethyl cellulose, calcium carboxymethyl cellulose, sodium carboxymethyl cellulose, starch, crystalline cellulose, starch, pregelatinized starch, and the like and mixtures thereof. The concentration of disintegrating agent ranges from about 1% to about 50% w/w of total composition, preferably from about 5% to about 30% w/w of total composition.

The terms “lubricants” or “glidants” or “anti-adherent agents” as used herein is defined as an agent for improving the fluidity of the powder and thus the filling of the granulation machine and the tablet press. The gliding agent may be present in the pharmaceutical composition of the present invention in the form of a single compound or in the form of a mixture of compounds. Suitable lubricants or glidants or anti-adherent agents include colloidal silicon dioxide (AEROSIL®), stearic acid, magnesium stearate, glyceryl monostearate, sodium stearyl fumarate, calcium stearate, talc, hydrogenated castor oil, sucrose esters of fatty acid, microcrystalline wax, yellow beeswax, white beeswax, and the like and mixtures thereof. The concentration of lubricant or glidant ranges from about 0.01% to about 10% w/w of total composition, preferably from about 0.5% to about 5% w/w of total composition.

As used herein, the term “granulating agents” or “granulating fluid” refers to any fluid composition which is capable of causing adhesion between ruxolitinib, binders and other intra-granular excipients. Suitable granulating fluids include aqueous, non-aqueous solvents or hydroalcoholic mixtures which collectively includes, but not limited to, water, ethanol, isopropanol (IPA), methanol, acetone or mixtures thereof. In hydro-alcoholic mixture, the weight ratio of aqueous to non-aqueous solvents is ranged from about 10:90 to about 90:10, preferably from about 20:80 to about 80:20.

The pharmaceutical compositions of the present invention may optionally contain coloring agent. The term “coloring agent” as used herein, refers to an agent which imparts colour to the formulation. Suitable coloring agent include dyes and pigments such as iron oxide red or yellow, titanium dioxide, talc. The concentration of coloring agent ranges from about 0.1% to about 10% w/w of total composition.

As used herein, “chelating agent” refers to an agent which forms via two or more of its functional groups, stable complexes with metal cations. Suitable chelating agents include, one or more of, but not limited to ethylenediaminetetraacetic acid (EDTA), disodium EDTA and derivatives thereof, citric acid and derivatives thereof, niacinamide and derivatives thereof, and sodium desoxycholate and the like or mixtures thereof. The concentration of chelating agent ranges from about 0.1% to about 20% w/w of total composition.

The pharmaceutical compositions of the present invention may additionally contain stabilizing agent. Suitable stabilizing agents include, one or more of, but not limited to α-tocopherol, butylated hydroxytoluene (BHT), butylated hydroxy anisole (BHA), tertbutylhydroquinone (TBHQ), propyl gallate (PG) and the like or mixtures thereof. The concentration of stabilizing agent ranges from about 0.1% to about 20% w/w of total composition.

The pharmaceutical composition may also optionally be coated, i.e., seal coated and/or film coated. Preferably, the pharmaceutical composition may be film coated. Suitable film-forming polymers are selected from the group comprising cellulose or its derivatives, e.g., hydroxypropyl cellulose, ethyl cellulose, methyl cellulose, carboxymethyl cellulose, hydroxymethyl cellulose, hydroxyethyl cellulose, cellulose acetate, hydroxypropyl methyl cellulose phthalate, cellulose acetate phthalate, and cellulose acetate trimellitate; waxes, methacrylic acid polymers, e.g., Eudragit®; and polyvinyl pyrrolidone. Alternatively, commercially available coating compositions comprising film-forming polymers marketed under various trade names, such as Opadry®, may also be used. Preferably, the film coating polymer may be present in an amount from about 0.1% to about 20% w/w of total composition.

In an embodiment, inventive pharmaceutical compositions comprising ruxolitinib and pharmaceutically acceptable excipients are prepared by using, but not limited, to wet granulation, dry granulation or direct compression and the resultant mixture is either compressed to tablet or filled in hard gelatin capsules.

The pharmaceutical composition of the present invention is preferably a granulate/particulate material. The granules/particles may be filled into a capsule or compressed into a tablet. The tablet may optionally be coated with an additional film coating. In a preferred embodiment, the inventive pharmaceutical composition is in the form of a tablet.

In a preferred embodiment, the present invention relates to extended-release pharmaceutical compositions of ruxolitinib that exhibit AUC₀₋ₜ within range of about 80% to 125% as the commercially available immediate release tablet formulation, but exhibits lower Cₘₐₓ. Such an inventive composition provides substantially the same clinical efficacy as commercially available ruxolitinib immediate release tablets, but improves side effect properties (reduced incidence of side effects). Furthermore, such extended-release formulations and methods of use will provide an opportunity to increase the daily dose of ruxolitinib while controlling the incidence of side effects. Furthermore, such extended-release formulations allow for the administration of ruxolitinib once-daily.

In an embodiment, extended-release composition of the present invention comprises a) ruxolitinib phosphate; and b) polyethylene oxide as release-controlling agent, wherein said composition upon oral administration in fasting or fed condition exhibits bioequivalence to reference composition (extended-release compositions comprising ruxolitinib and a release-controlling agent), in fasting or fed condition, and wherein said bioequivalence is established by at least one parameter that is selected from (i) a confidence interval for mean AUC₀₋ₜ between about 80% and about 125%; (ii) a confidence interval for mean AUC_{0-infinity} between about 80% and about 125%; (iii) a confidence interval for mean Cₘₐₓ between about 80% and about 125% or combination thereof.

In an embodiment, extended-release composition of the present invention comprises a) ruxolitinib phosphate; and b) polyethylene oxide as release-controlling agent, wherein said composition upon oral administration in fasting or fed condition exhibits bioequivalence to reference composition (extended-release compositions comprising ruxolitinib and hydroxypropyl methyl cellulose as release-controlling agent), in fasting or fed condition, and wherein said bioequivalence is established by at least one parameter that is selected from (i) a confidence interval for mean AUC₀₋ₜ between about 80% and about 125%; (ii) a confidence interval for mean AUC_{0-infinity} between about 80% and about 125%; (iii) a confidence interval for mean Cₘₐₓ between about 80% and about 125% or combination thereof.

In some embodiments, the present invention provides ruxolitinib extended-release pharmaceutical compositions exhibiting following in-vitro drug release profile: within 2 hours, not less than 20% of the total ruxolitinib is released; within 3 hours, not more than 70% of the total ruxolitinib is released, wherein the composition provides therapeutically effective plasma concentration of ruxolitinib over a period of 24 hours to treat myelofibrosis, polycythemia vera, acute graft-versus-host disease (aGVHD) and chronic graft-versus-host disease (cGVHD).

Stability: Ruxolitinib has known as well as unknown impurities. In particular, amide impurity, acid impurity, SEM pyrimidine impurity and acrylo pyrimidine impurities were monitored. The chemical names, structures and origin of these impurities are provided below:

| Impurity name | Chemical name | Structure | Origin |
|---|---|---|---|
| Ruxolitinib amide, Amide impurity, Ruxolitinib impurity-6 | 3-(4-(7H-Pyrrolo[2,3- d]pyrimidin-4-yl)-1H- pyrazol-1-yl)-3- cyclopentylpropanamide | 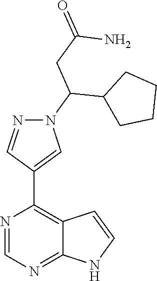 [SMILES: NC(=O)CC(C1CCCC1)n1cc(-c2ncnc3[nH]ccc23)cn1] | Degradation |
| Ruxolitinib acid, Acid impurity, Ruxolitinib impurity-8 | (R)-3-(4-(7H-pyrrolo[2, 3-d]pyrimidin-4-yl)-1H- pyrazol-1-yl)-3- cyclopentylpropanoic acid | 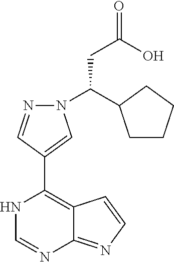 [SMILES: O=C(O)C[C@H](C1CCCC1)n1cc(-c2[nH]cnc3nccc2-3)cn1] | Degradation |
| SEM Pyrimidine impurity | 4-(1H-pyrazol-4-yl)-7- ((2-(trimethylsilyl) ethoxy)methyl)-7H- pyrrolo[2,3-d] pyrimidine | 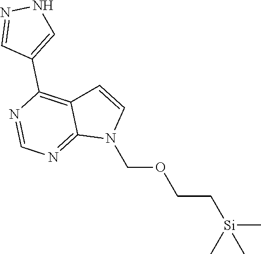 [SMILES: C[Si](C)(C)CCOCn1ccc2c(-c3cn[nH]c3)ncnc21] | Process |
| Acrylopyrimidine impurity | 3-cyclopentyl-3-(4-(7- ((2-(trimethylsilyl) ethoxy)methyl)-7H- pyrrolo[2,3-d]pyrimidin- 4-yl)-1H-pyrazol-1- yl)propanenitrile | 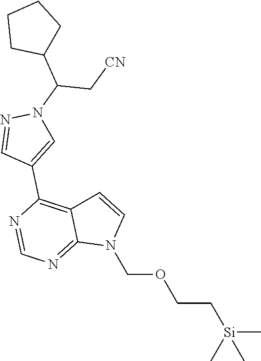 [SMILES: C[Si](C)(C)CCOCn1ccc2c(-c3cnn(C(CC#N)C4CCCC4)c3)ncnc21] | Process |

Methods of treatment: Ruxolitinib is a JAK inhibitor. JAK plays a central role in a number of cytokine and growth factor signaling pathways that, when dysregulated, can result in or contribute to disease states.

Another aspect of the present invention pertains to methods of treating a JAK-associated disease or disorder in an individual (e.g., patient) by administering an extended-release composition of the present invention to the individual in need of such treatment. A JAK-associated disease can include any disease, disorder or condition that is directly or indirectly linked to expression or activity of the JAK, including overexpression and/or abnormal activity levels. A JAK-associated disease can also include any disease, disorder or condition that can be prevented, ameliorated, or cured by modulating JAK activity.

Dosage and Administration: The recommended starting dose of ruxolitinib is based on platelet count. Treatment with ruxolitinib can cause hematologic adverse reactions, including thrombocytopenia, anemia and neutropenia. A complete blood count (CBC) and platelet count must be performed before initiating therapy, every 2 to 4 weeks until doses are stabilized, and then as clinically indicated. Doses may be titrated based on safety and efficacy. The dose of the therapeutic compound will be in the range from about 1 mg to about 300 mg once-daily. Exemplary unit dosage form of ruxolitinib on free bases range from 1 mg to 300 mg, including unit dosages of 1 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 120 mg, 140 mg, 160 mg, 180 mg, 200 mg, 225 mg, 250 mg, 275 mg and 300 mg.

### Analysis of Samples Withdrawn During Chemical Analysis of Ruxolitinib

The samples withdrawn were analyzed for drug content using the following HPLC procedure. The materials and general conditions are listed below:

TABLE 1
Chromatographic conditions (Drug content & Dissolution)

| Chromatographic Mode | HPLC system equipped with UV/PDA detector | |
|---|---|---|
| Column | Xterra RP18 150 × 4.6 mm, 5 μm (Part # 186000492) | |
| Wavelength | 294 | nm |
| Flow rate | 1.0 | mL/minute |
| Injection volume | 10 | μL |
| Column temperature | 30° C. ± 2° C. | |
| Sample temperature | 25° | C. |
| Run time | 10 | minutes |

| | |
|---|---|
| Mobile Phase | Mobile Phase buffer: Transfer 6.8 g of Potassium dihydrogen |
| | phosphate anhydrous into 1000 mL of water and sonicate to dissolve |
| | and adjust the pH of solution to 2.50 ± 0.05 with |
| | ortho-Phosphoric acid and filter through 0.45 μ membrane filter. |
| | Mobile Phase: Mix well mobile phase buffer, Methanol |
| | and Acetonitrile in a ratio of 60:20:20 v/v, sonicate to degas. |
| Mode of Elution | Isocratic |

The samples withdrawn were analyzed for related substances using the following HPLC procedure. The materials and general conditions are listed below:

TABLE 2
Chromatographic conditions (Related substance analysis)

| Chromatographic Mode | HPLC system equipped with UV/PDA detector | | |
|---|---|---|---|
| Column | Inertsil ODS3V, 150 × 4.6 mm, 5 μm (C/N: 5020-01801) | | |
| Wavelength | 225 | | nm |
| Flow rate | 1.0 | | mL/minute |
| Injection volume | 10 | | μL |
| Column temperature | 25° | | C. |
| Sample temperature | 20° | | C. |
| Run time | 60 | | minutes |
| Mobile Phase A | Transfer 1.36 g of Potassium dihydrogen phosphate | | |
| | anhydrous into 2000 mL of water and sonicate to | | |
| | dissolve and adjust the pH of solution to 2.80 ± 0.05 with | | |
| | ortho-Phosphoric acid and filter through 0.45 μ membrane filter. | | |
| Mobile Phase B | Mix 800 mL of Acetonitrile and 200 mL of Mobile phase- | | |
| | A and sonicate to degas. | | |
| | Gradient | | |
| | Time | Mobile Phase-A (%) | Mobile Phase-B (%) |
| Mode of Elution | 0.01 | 90 | 10 |
| | 5 | 90 | 10 |
| | 20 | 60 | 40 |
| | 28 | 60 | 40 |
| | 35 | 40 | 60 |
| | 40 | 0 | 100 |
| | 50 | 0 | 100 |
| | 52 | 90 | 10 |
| | 60 | 90 | 10 |

### EXAMPLES

The following examples are exemplary and not intended to be limiting. The above disclosure provides many different embodiments for implementing the features of the invention, and the following examples describe certain embodiments. It will be appreciated that other modifications and methods known to one of ordinary skill in the art can also be applied to the following experimental procedures, without departing from the scope of the invention.

### Example 1

Ruxolitinib compositions were set forth in Table 3

TABLE 3

| | |
|---|---|
| Composition | Composition |
| A | B |

| | |
|---|---|
| Ingredients | Quantity (in mg)/unit |

Intra-granular ingredients:

| | | |
|---|---|---|
| Ruxolitinib phosphate | 66.00 | 66.00 |
| Microcrystalline cellulose | 343.95 | 346.65 |
| Polyethylene oxide | 108.00 | 108.00 |
| (POLYOX ™ WSR) | | |

Binder solution (IPA: Water: 95:5) - 15% w/w of dry mix
Extra-granular ingredients:

| | | |
|---|---|---|
| Colloidal Silicon Dioxide | 5.40 | 5.40 |
| BHT | 0.45 | 0.45 |
| Magnesium Stearate | 5.40 | 2.70 |
| Stearic Acid | 10.80 | 10.80 |
| Core tablet weight | 540.00 | 540.00 |

Coating (3% w/w build up)

| | | |
|---|---|---|
| Opadry II white 85F18422 | 16.2 | — |
| Water | Q.S. | — |
| Coated tablet weight (mg) | 556.20 | 540.00 |

### Manufacturing Procedure for Composition A & B:

All the materials were dispensed as per above batch formula. Intra-granular material was sifted and mixed in a granulator. Mix was granulated with the binder solution. Wet granules were dried and sifted. All the sifted granules were mixed with extra-granular material and lubricated with a lubricant. Lubricated blend was compressed and later coated (if applicable). Samples of Composition A were stored in HDPE containers at 40° C./75% RH and 25° C./60% RH for stability evaluation.

TABLE 4
Dissolution profile of Composition A and B
Condition: 0.1N HCl media, USP-II, 500 mL, 50 rpm at 37° C.

| | Composition A | | | |
|---|---|---|---|---|
| | | 40° C./75% RH | 25° C./60% RH | Composition B |
| Time | Initial | 6 months | 6 months | Initial |
| (hour) | % Drug release | | | |
| 0.5 | 18 | 20 | 19 | 22 |
| 1 | 28 | 30 | 28 | 31 |
| 2 | 43 | 46 | 44 | 45 |
| 3 | — | 57 | 56 | — |
| 4 | 66 | 67 | 66 | 65 |
| 6 | — | 80 | 80 | 79 |
| 7 | 90 | 90 | 90 | — |
| 8 | — | — | — | 87 |
| 12 | 100 | 99 | 100 | 96 |

Composition A exhibits drug release profile of not less than 20% of total amount of ruxolitinib is released within 2 hours and not more than 60% of total amount of ruxolitinib is released within 3 hours.

TABLE 5
Dissolution profile of Composition A

| Time | Composition A |
|---|---|
| (hour) | % Drug release |
| Dissolution | 0.1N HCl media, USP-II, 500 mL, 75 rpm at |
| media | 37° C. |
| 1 | 52 |
| 2 | 60 |
| Dissolution | pH 6.8 Phosphate Buffer media, USP-II, |
| media change | 900 mL, 75 rpm at 37° C. |
| 3 | 64 |
| 4 | 73 |
| 5 | 93 |
| 6 | 98 |

TABLE 6
Stability of Composition A

| | | 40° C./75% RH | 25° C./60% RH |
|---|---|---|---|
| Parameter | Initial | 6 months | 6 months |
| Assay (%) | 101.5 | 102 | 102.5 |

Related substances (%)

| | | | |
|---|---|---|---|
| Amide impurity (RRT 0.78) | ND | 0.02 | 0.03 |
| Acid impurity (RRT 0.90) | ND | ND | ND |
| Highest unknown | 0.04 | 0.03 | 0.03 |
| Total impurities | 0.19 | 0.14 | 0.12 |

Stability data for Composition A was satisfactory with no significant change in assay, impurity profile when stored for 6 months at 40° C./75% RH and 25° C./60% RH conditions.

### Example 2

Ruxolitinib compositions were set forth in Table 7

TABLE 7

| | |
|---|---|
| Composition C | Composition D |

| | |
|---|---|
| Ingredients | Quantity (in mg)/unit |

Intra-granular ingredients:

| | | |
|---|---|---|
| Ruxolitinib Phosphate | 66.00 | 66.00 |
| Microcrystalline Cellulose | 400.65 | 292.65 |
| Polyethylene oxide | 54.00 | 162.00 |
| (POLYOX ™ WSR 301 LEO) | | |

Binder (IPA: Water: 95:5) - 15% w/w of dry mix
Extra-granular ingredients:

| | | |
|---|---|---|
| Colloidal Silicon Dioxide | 5.40 | 5.40 |
| BHT | 0.45 | 0.45 |
| Magnesium Stearate | 2.70 | 2.70 |
| Stearic Acid | 10.80 | 10.80 |
| Tablet weight | 540.00 | 540.00 |

### Manufacturing Procedure for Composition C and D:

All the materials were dispensed as per above batch formula. Intra-granular material was sifted and mixed in a granulator. Mix was granulated with the binder solution. Wet granules were dried and sifted. All the sifted granules were mixed with extra-granular material and lubricated with a lubricant. Lubricated blend was compressed.

TABLE 8
Dissolution Data of composition C and D:
Dissolution media and parameters:
0.1N HCl media, USP-II, 500 mL, 50 rpm at 37° C.

| Time | Composition C | Composition D |
|---|---|---|
| (hour) | % Drug release | % Drug release |
| 0.5 | 16 | 11 |
| 1 | 24 | 16 |
| 2 | 38 | 25 |
| 4 | 54 | 38 |
| 6 | 64 | 48 |
| 8 | 70 | 57 |
| 12 | 78 | 69 |

Composition C and D exhibits drug release profile of not less than 20% of total amount of ruxolitinib is released within 2 hours and not more than 60% of total amount of ruxolitinib is released within 3 hours.

### Example 3

Ruxolitinib compositions were set forth in Table 9

TABLE 9

| | |
|---|---|
| | Composition E |
| Ingredients | Quantity (in mg)/unit |

Intra-granular ingredients:

| | |
|---|---|
| Ruxolitinib Phosphate | 66.00 |
| Microcrystalline Cellulose | 316.95 |
| POLYOX ™ WSR 301 LEO | 108.00 |

Binder (IPA: Water: 80:20) - 30% w/w of dry mix

| | |
|---|---|
| Polyvinyl pyrrolidine | 27.00 |
| Isopropyl alcohol | Q.S. |
| Water | Q.S. |

Extra-granular ingredients:

| | |
|---|---|
| Colloidal Silicon Dioxide | 5.40 |
| BHT | 0.45 |
| Magnesium Stearate | 2.70 |
| Stearic Acid | 10.80 |
| Core tablet weight | 540.00 |

### Manufacturing Procedure for Composition E:

All the materials were dispensed as per above batch formula. Intra-granular material was sifted and mixed in a granulator. Mix was granulated with the binder solution. Wet granules were dried and sifted. All the sifted granules were mixed with extra-granular material and lubricated with a lubricant. Lubricated blend was compressed.

TABLE 10
Dissolution Data of Composition E
Dissolution media and parameters:
0.1N HCl media, USP-II, 500 mL, 50 rpm at 37° C.

| Time (hour) | % Drug release |
|---|---|
| 0.5 | 13 |
| 1 | 21 |
| 2 | 33 |
| 4 | 48 |
| 6 | 58 |
| 8 | 65 |
| 12 | 73 |

Composition E exhibits drug release profile of not less than 20% of total amount of ruxolitinib is released within 2 hours and not more than 60% of total amount of ruxolitinib is released within 3 hours.

### Example 4

Ruxolitinib compositions were set forth in Table 11

TABLE 11

| | |
|---|---|
| Composition F | Composition G |

| | |
|---|---|
| Ingredients | Quantity (in mg)/unit |

Intra-granular excipients:

| | | |
|---|---|---|
| Ruxolitinib Phosphate | 66.00 | 66.00 |
| Microcrystalline Cellulose | 347.10 | 346.88 |
| POLYOX ™ WSR 301 LEO | 108.00 | 108.00 |

Binder solution (IPA: Water: 95:5)

| | | |
|---|---|---|
| Isopropyl alcohol | Q.S. | Q.S. |
| Water | — | Q.S. |

Extra-granular ingredients:

| | | |
|---|---|---|
| Colloidal Silicon Dioxide | 5.40 | 5.40 |
| BHT | — | 0.22 |
| Magnesium Stearate | 2.70 | 2.70 |
| Stearic Acid | 10.80 | 10.80 |
| Core tablet weight | 540.00 | 540.00 |

TABLE 12
Stability & Dissolution data of Composition F

| | | | |
|---|---|---|---|
| | | 40° C./75% RH | 25° C./60% RH |
| Parameter | Initial | 3 months | 3 months |

Related substances (%)

| | | | |
|---|---|---|---|
| Amide impurity | ND | 0.01 | 0.02 |
| Highest unknown | 0.31 | 0.69 | 0.88 |
| Total impurities | 0.31 | 1.63 | 1.86 |

Dissolution parameters (0.1N HCl, 500 mL, 50 rpm, USP-II) at 37° C.

| Time (in hours) | % Drug release | | |
|---|---|---|---|
| 0.5 hour | 12 | 18 | 14 |
| 1 hour | 20 | 25 | 21 |
| 2 hours | 31 | 40 | 33 |
| 4 hours | 48 | 60 | 52 |
| 6 hours | 60 | 75 | 65 |
| 8 hours | 69 | 84 | 75 |
| 12 hours | 80 | 89 | 88 |

TABLE 13
Stability of Composition G

| | Composition G | |
|---|---|---|
| | Initial | 40° C./75% RH |
| Parameter | — | 6 months |
| Assay (%) | 96.5 | — |

Related substances (%)

| | | |
|---|---|---|
| Amide impurity | ND | 0.12 |
| Acid impurity | ND | ND |
| Highest unknown (RRT at 0.58) | 0.05 | 0.04 |
| Total impurities | 0.12 | 0.16 |

Dissolution (0.1N HCl, 500 mL, 50 rpm, USP-II)

| Time (in hours) | % Drug release | |
|---|---|---|
| 0.5 hour | 12 | 12 |
| 1 hour | 19 | 20 |
| 2 hours | 31 | 32 |
| 4 hours | 48 | 49 |
| 6 hours | — | 62 |
| 8 hours | 72 | 72 |
| 12 hours | 86 | 84 |

Composition F without stabilizing agent (i.e., BHT) showed increase in Highest unknown impurity at initial and more than 1.5% w/w of total impurities after storage for 3 months at 40° C./75% RH & 25° C./60% RH. The level of total impurities in Composition G containing stabilizing agent (i.e., BHT) was less than 1.5% w/w of total composition when stored for 6 months at 40° C./75% RH.

### Example 5

Comparative composition of ruxolitinib were set forth in Table 14

TABLE 14
Comparative Composition

| | |
|---|---|
| | Quantity (in |
| Ingredients | mg)/unit |

Intra-granular Excipients

| | |
|---|---|
| Ruxolitinib Phosphate | 66.00 |
| Microcrystalline Cellulose (Avicel PH 101) | 118.35 |
| Methocel ™ K15M | 21.60 |
| Methocel ™ K4M | 86.40 |
| Lactose Monohydrate | 228.30 |
| Water | Q.S. |

Extra-granular Excipients

| | |
|---|---|
| Colloidal Silicon Dioxide (Aerosil ® 200 pharma) | 5.40 |
| Butylated Hydroxy Toluene (BHT) | 0.45 |
| Magnesium Stearate (Ligamed-MF-2-V) | 2.70 |
| Stearic Acid (Kolliwax S fine) | 10.80 |

Core Tablet Weight

| | |
|---|---|
| Opadry II Complete film coating system 85F18422 White | 16.20 |
| Water | Q.S. |

Coated Tablet Weight

What is claimed is:

1. A monolithic, matrix-based, extended-release pharmaceutical composition suitable for once-daily administration, said composition comprising:
a) ruxolitinib phosphate; and
b) a release-controlling polymer, which is polyethylene oxide;
wherein not less than 20% of total amount of ruxolitinib is released from said composition within 2 hours, and not more than 60% of total amount of ruxolitinib is released from said composition within 3 hours as determined by USP dissolution apparatus II (paddle) at 50 rpm in 500 ml of 0.1N HCI as dissolution medium; and
wherein the weight of polyethylene oxide in the pharmaceutical composition is not more than 30% by weight of the composition.

2. The composition of claim 1, further comprising at least one stabilizing agent.

3. The composition of claim 1, wherein a level of total impurities in said composition is less than 2.0% w/w when stored at 25° C./60% RH for at least 3 months.

4. The composition of claim 1, wherein said composition does not contain hydroxypropyl methyl cellulose as the release-controlling polymer.

5. The composition of claim 1, wherein said composition comprises: an intra-granular phase containing at least said ruxolitinib phosphate and said polyethylene oxide; and an extra-granular phase containing at least one additional excipient selected from diluents, binders, chelating agents, coating agents, disintegrating agents, lubricants, glidants, colorants, surfactants, plasticizers, or mixtures thereof.

6. The composition of claim 5, wherein said intra-granular phase further comprises microcrystalline cellulose.

7. The composition of claim 5, wherein said extra-granular phase comprises magnesium stearate as a lubricant.

8. The composition of claim 1, further comprising a diluent selected from group consisting of microcrystalline cellulose, silicified microcrystalline cellulose, calcium carbonate, calcium sulfate, cellulose powdered, dextrates, dextrins, dextrose, fructose, kaolin, lactitol, lactose, starch, starch pregelatinized and sucrose.

9. The composition of claim 8, wherein the amount of said diluent ranges from about 60% to about 95% w/w of total composition.

10. The composition of claim 1, wherein said composition is a tablet.

11. The composition of claim 1, wherein said composition is prepared using wet granulation.

12. The composition of claim 1, wherein said ruxolitinib phosphate is present in an amount of 13.2 mg, or 26.4 mg, or 39.6 mg, or 52.8 mg, or 66 mg, or 79.2 mg.

13. A method of treating a condition selected from myelofibrosis, polycythemia vera, aGVHD, or cGVHD, wherein said method comprises administering the composition of claim 1 to a patient in need thereof.

14. The composition of claim 1, wherein not more than 70% of total amount of ruxolitinib is released from said composition within 3 hours as determined by USP dissolution apparatus II (paddle) at 75 rotations per minute (rpm) in 900 ml of re-buffered dissolution medium to pH 6.8.

15. A monolithic, matrix-based, extended-release pharmaceutical composition suitable for once-daily administration, said composition comprising:
a) ruxolitinib phosphate; and
b) a release-controlling polymer, which is polyethylene oxide;
wherein not more than 40% of total amount of ruxolitinib is released from said composition within 1 hour, 40% to 70% of total amount of ruxolitinib is released from said composition within 3 hours, and not less than 80% of total amount of ruxolitinib is released from said composition within 12 hours, as determined by USP dissolution apparatus II (paddle) at 50 rpm in 500 mL of 0.1N HCI as dissolution medium.

16. The composition of claim 15, further comprising at least one stabilizing agent.

17. The composition of claim 15, wherein a level of total impurities in said composition is less than 2.0% w/w when stored at 25° C./60% RH for at least 3 months.

18. The composition of claim 15, wherein said composition comprises: an intra-granular phase containing at least said ruxolitinib phosphate and said polyethylene oxide; and an extra-granular phase containing at least one additional excipient selected from diluents, binders, chelating agents, coating agents, disintegrating agents, lubricants, glidants, colorants, surfactants, plasticizers, or mixtures thereof.

19. The composition of claim 18, wherein said intra-granular phase further comprises microcrystalline cellulose.

20. The composition of claim 18, wherein the weight of polyethylene oxide in the pharmaceutical composition is not more than 30% by weight of the composition.
